(19) United States
(12) Patent Application Publication
(10) Pub. No.: US 2026/0298815 A1
(43) Pub. Date: Oct. 1, 2026

(54) HYPERSPECTRAL IMAGING ANALYTICAL METHOD FOR DETERMINING TOBACCO COMPONENTS AND LEVELS THEREOF

(71) Applicant: BRITISH AMERICAN TOBACCO (INVESTMENTS) LIMITED, London (GB)

(72) Inventors: Marcelo Caetano Alexandre MARCELO, São Leopoldo (BR); Maurilio Gustavo NESPECA, Porto Alegre (BR); Jailson Cardoso DIAS, Belém (BR); Jorge Armando Ardila QUINTERO, Santa Cruz do Sul (BR); Samuel KAISER, Porto Alegre (BR); Priscila CRUZ, Canoas (BR)

(21) Appl. No.: 19/476,357

(22) PCT Filed: Apr. 19, 2024
(86) PCT No.: PCT/EP2024/060828

§ 371 Date: Oct. 17, 2025

(30) Foreign Application Priority Data
Apr. 19, 2023 (BR) 1020230074138
Apr. 19, 2023 (GB) 2305771.4

(51) Int. Cl. G01N 21/3563; G01N 21/359; G01N 33/00

(52) U.S. Cl. CPC G01N 21/3563; G01N 21/359; G01N 33/0098

(57) ABSTRACT

A computer implemented method of determining a property of an unknown tobacco sample comprising using hyperspectral imaging in combination with a machine-learning-based prediction framework. The method comprises: creating a first, reference spectral fingerprint dataset by scanning a plurality of known tobacco samples using hyperspectral imaging techniques, generating hyperspectral images of each of said known tobacco samples and forming a reference spectral fingerprint, and storing each of said reference spectral fingerprints into said first, reference spectral fingerprint dataset. The method also comprises providing a second, known property, dataset, comprising known properties/compositions of said known tobacco sample, and inputting said first, reference spectral fingerprint dataset and said second, known property, dataset into said machine learning-based prediction framework. The method further comprises via said machine learning-based prediction framework, correlating said first, spectral fingerprint dataset with said known property or properties of said second, known property, dataset; said machine-learning based prediction framework generating a prediction model that is configured to determine one or more mathematical functions based on said first reference spectral fingerprint dataset, and said second, known property dataset. The method further comprises generating hyperspectral images of said unknown tobacco sample, and based on said hyperspectral images of said unknown tobacco sample, generating a spectral fingerprint of said unknown tobacco sample. The method also comprises inputting said spectral fingerprint of said unknown tobacco sample into said machine learning-based prediction framework, and said machine-learning-based prediction framework applying said one or more mathematical functions to obtain and output said determined property of said unknown tobacco sample.

### CROSS-REFERENCE TO RELATED APPLICATIONS

This present application is a National Phase entry of PCT Application No. PCT/EP2024/060828, filed Apr. 19, 2024, which claims priority from Brazilian application Ser. No. 1020230074138, filed Apr. 19, 2023, and which claims priority from Great Britain Application No. 2305771.4, filed Apr. 19, 2023, each of which are fully incorporated herein by reference in their entireties.

### TECHNICAL FIELD

Methods and systems for determining properties, components and/or sensory attributes of an unknown tobacco sample are described herein.

### BACKGROUND

Sensory evaluation plays an important role in the assessment of tobacco quality. The determination of tobacco sensory attributes can be used for product quality control, crop consistency ensuring, blend designs, estimation of product shelf-life, among other applications. Human sensory analysis is the main method that is currently used for evaluating tobacco product quality and consumer acceptance.

### SUMMARY

In accordance with some embodiments described herein, there is provided a computer implemented method of determining a property or properties of an unknown tobacco sample. The method uses hyperspectral imaging in combination with a machine-learning-based prediction framework.

The method comprises creating a first, reference spectral fingerprint dataset by scanning a plurality of known tobacco samples using hyperspectral imaging techniques, generating hyperspectral images of each of said known tobacco samples and storing each of said reference spectral fingerprints into said first, reference spectral fingerprint dataset.

The method further comprises providing a second, known property, dataset, comprising known properties of said known tobacco sample, and inputting said first, reference spectral fingerprint dataset and said second, known property, dataset into said machine learning-based prediction framework.

The method further comprises, via said machine learning-based prediction framework, correlating said first, spectral fingerprint dataset with said known property or properties of said second, known property, dataset.

The method further comprises generating a prediction model that is configured to determine one or more mathematical functions based on said first, reference, spectral fingerprint dataset and said second, known property, dataset.

The method further comprises using a, or said, hyperspectral imaging camera to scan said unknown tobacco sample and generating hyperspectral images of said unknown tobacco sample, and based on said hyperspectral images of said unknown tobacco sample, generating a spectral fingerprint of said unknown tobacco sample.

The method further comprises inputting said spectral fingerprint of said unknown tobacco sample into said machine learning-based prediction framework, and said machine-learning-based prediction framework applying said one or more mathematical functions of said prediction model to said spectral fingerprint of said unknown tobacco sample to obtain and output said determined property of said unknown tobacco sample based on said prediction model.

The one or more mathematical functions may comprise any suitable function. In some embodiments, the one or more mathematical functions may comprise regression coefficients.

In some embodiments, the outputted property or properties may comprise one or more of a chemical compound, a toxicant, determination of leaf position, leaf colour and/or leaf quality, taste profile and/or a sensory attribute of said unknown tobacco sample.

The outputted properties may also or alternatively comprise other properties and are not restricted to only those listed here.

The second, known property, dataset may be related to known properties and/or composition(s) of said known tobacco samples. These known properties may be sensory attributes, taste profile, leaf position, leaf colour and/or leaf quality that may be determined by human assessment or by an indirect instrumental method obtained in a laboratory. In some embodiments the composition may be related to concentration levels of chemical compounds determined by analytical instrumental methods in a laboratory. The dataset is not, however, limited to only these properties but other properties may also be used.

In accordance with some embodiments described herein, there is also provided a computer implemented method of training a machine learning-based prediction framework for determining a property or properties of an unknown tobacco sample.

The method comprises: creating a first, reference spectral fingerprint dataset by scanning a plurality of known tobacco samples using hyperspectral imaging techniques, generating hyperspectral images of each of said known tobacco samples and forming a reference spectral fingerprint of each of said known samples.

The method further comprises creating a second, known property, dataset, relating to a known property and/or composition of said known tobacco sample, and inputting said first and second datasets into said machine learning-based prediction framework.

The method further comprises, via said machine-learning based prediction framework correlating said first, spectral fingerprint dataset with said known property or properties of said second, known property, dataset and generating a prediction model based on said first and said second datasets.

The method further comprises using a hyperspectral imaging camera to scan said unknown tobacco sample and generating hyperspectral images of said unknown tobacco sample, and based on said hyperspectral images of said unknown tobacco sample, generating a spectral fingerprint of said unknown tobacco sample, inputting said spectral fingerprint of said unknown tobacco sample into said machine learning-based prediction framework, and said machine-learning-based prediction framework applying said prediction model to said spectral fingerprint of said unknown tobacco sample to obtain and output said determined property of said unknown tobacco sample based on said prediction model. The properties comprise one or more of a chemical compound, a toxicant, determination of leaf position, leaf colour and/or leaf quality, taste profile and/or a sensory attribute of said unknown tobacco sample.

The step of correlating may further comprise correlating said property of each of said reference spectral fingerprints with one or more of a chemical compound, a toxicant, determination of a leaf position, leaf colour, leaf quality, and/or a sensory attribute a sensory attribute.

The second dataset is related to known properties and/or composition(s) of the known tobacco samples. These known properties may be sensory attributes, taste profile, leaf position, leaf colour and/or leaf quality that may be determined by human assessment or by an indirect instrumental method obtained in a laboratory. The composition may be related to concentration levels of chemical compounds determined by analytical instrumental methods in a laboratory.

The prediction model that is generated based on both the first and second datasets is configured to determine one or more mathematical functions (e.g. regression coefficients) based on both of said datasets.

The machine-learning-based prediction framework may apply said one or more mathematical functions (e.g. regression coefficients) of said prediction model to said spectral fingerprint of said unknown tobacco sample to obtain and output said determined property of said unknown tobacco sample based on said prediction model, wherein said properties comprise one or more of a chemical compound, a toxicant, determination of leaf position, leaf colour and/or leaf quality, taste profile and/or a sensory attribute of said unknown tobacco sample.

In any of the embodiments described herein, the method may further comprise said prediction model chemically grading said unknown tobacco sample based on said predicted property.

In any of the embodiments described herein, the method may further comprise said prediction model segregating said unknown tobacco sample based on said predicted property.

In any of the embodiments described herein, the method may further comprise said prediction model identifying, analysing and/or quantifying chemicals contained within said unknown tobacco sample based on said predicted property.

In any of the embodiments described herein, the method may further comprise said prediction model segregating said unknown tobacco sample based on said chemical, toxicant, leaf position, leaf colour and/or leaf quality, taste profile and/or a sensory attribute generated by prediction models.

In any of the embodiments described herein, the method may further comprise said machine-learning based prediction framework utilising linear algorithms.

In any of the embodiments described herein, the method may further comprise said machine learning based prediction framework utilising non-linear algorithms.

In any of the embodiments described herein, the method may further comprise said machine-learning based prediction framework being a neural network.

In any of the embodiments described herein, the method may be implemented using a numerical computing environment and/or framework.

In any of the embodiments described herein, the numerical computing environment and/or framework may comprise using any IDE (Integrated Development Environment). Examples of these may comprise Pycharm, Spyder, Jupiter, R Studio; or any Numerical-software packages, or any of MATLAB, GNU Octave, Pycharm, and R Studio. The embodiments described herein are not limited to these examples, however.

In any of the embodiments described herein, the method may be implemented using one or more programming languages.

In any of the embodiments described herein, said one more programming languages may comprise any general purpose programming language, for example any one or more of Python, C, C++, C#, R, Julia and Java. The embodiments described herein are not limited to these examples, however.

A machine learning-based prediction framework is also described that is produced by the method of any preceding claim.

A computer system for producing a machine learning-based prediction framework is also described, wherein the computer system is configured to perform any of the methods described herein.

In any of the embodiments described herein, the prediction model developed by said machine-learning based prediction framework may be configured to chemically grade said unknown tobacco sample based on said predicted chemical quantification, or sensory graded by predicted sensory attributes, or chemically and sensory graded using both information.

In any of the embodiments described herein, the prediction model developed by said machine-learning based prediction framework may be configured to segregate said unknown tobacco sample based on said predicted sensory attribute.

In any of the embodiments described herein, the prediction model developed by said machine-learning based prediction framework may be configured to identify, analyse and quantify chemicals contained within said unknown tobacco sample based on said spectral fingerprint of the unknown tobacco sample.

In any of the embodiments described herein, the prediction model developed by said machine-learning based prediction framework may be configured to segregate said unknown tobacco sample based on said chemical quantification.

In any of the embodiments described herein, said prediction model developed by said machine-learning based prediction framework may be configured to chemically grade said unknown tobacco sample by predicted chemicals quantification, or sensory graded by predicted sensory attributes, or chemically and sensory graded using both information.

In any of the embodiments described herein, said prediction model developed by said machine-learning based prediction framework may be configured to segregate said unknown tobacco sample based on said predicted property.

In any of the embodiments described herein, said prediction model developed by said machine-learning based prediction framework may be configured to identify, analyse and quantify chemicals contained within said unknown tobacco sample based on said spectral fingerprint of the unknown tobacco sample.

### BRIEF DESCRIPTION OF THE DRAWINGS

Embodiments of the present invention will now be described, by way of example only, with reference to the accompanying drawings.

FIG. 1 depicts the installation of a HSI camera in a buying channel.

FIG. 2 depicts the installation of a HSI camera in a packing channel.

FIG. 3 depicts pre-processed spectra of an unknown tobacco sample from a buying channel, i.e. a spectral fingerprint

FIG. 4 depicts pre-processed spectra of an unknown tobacco sample from a packing channel.

FIG. 5 depicts an example of flow of data in a new method and system for analysing a tobacco sample as described herein.

### DETAILED DESCRIPTION

The systems and methods described herein may provide an inline methodology for the determination of tobacco properties and/or sensory attributes through a combination of hyperspectral imaging and artificial intelligence and machine learning. The new approaches described herein are based on computer implemented analytical methods that can provide many advantages to tobacco sensory evaluation in comparison to methods that are currently used.

Additionally, or alternatively, the systems and methods described herein may use this methodology to determine other properties of the tobacco sample, to detect and determine specific components and levels thereof of the tobacco sample. The methods and systems described herein may additionally or alternatively be used for the determination of leaf position, leaf colour and/or leaf quality.

The new approaches described herein correlate the chemical information of the tobacco samples being analysed with human sensory responses through the use of a computer-implemented machine-learning method that uses multivariate algorithms. The resulting output information is similar to human sensory assessment, but much faster, less expensive, more robust and reproducible.

Image processing techniques associated with multivariate algorithms has increasingly become a promising tool for precise and real-time process control. Infrared hyperspectral image systems (HSI) are the combination of image processing techniques with analytical chemistry. Therefore, via the use of HSI systems, not only a visual inspection is provided through an image but also the chemical information of a product is determined and can then be controlled.

Moreover, HSI systems and methods meet the main requirements for inline assessment of tobacco quality: non-destructiveness, real-time information, robustness and reproducibility.

The examples described herein provide an analytical platform which combines real time chemical analysis, machine learning algorithms and software for the management, grading, chemical composition quantitation and sensory profile determination of tobacco.

The analytical platform further: a) is able to provide superficial chemical analysis of tobacco (bales, bundles, leaves, etc.) through a HSI system; b) is able to perform a plurality of image preprocess steps and machine learning algorithms to quantify chemical contents such as nicotine, sugar, tobacco specific nitrosaminas, nitrates, and others, as well as to determine the sensory profile in terms of attributes and taste profile, and classify the tobacco based on these characteristics; and c) is able to provide reports, management and class creation tools based on these chemical contents.

This analytical platform can be implemented in any process step of tobacco production or cigarette manufactory inside its application scope and provide real time responses.

In this way, the examples described herein are able to provide a real time system for tobacco sensory and chemical assessment. The system and method are configured to translate hyperspectral images into tobacco properties, with 56 different tobacco properties being able to be determined. The system and method are not limited to this number, however, and further properties may also be determined.

The infrared imaging system used and described herein is configured to be able to process at least around 1200 images in one hour. As described in detail below, the systems and methods described herein use machine learning in combination with infrared imaging in order to provide an assessment of a tobacco product that is safe and in line with data protection. The systems and methods are able to utilise this combination of infrared imaging with machine learning to output an assessment of the tobacco, which provides information regarding the chemicals, toxicants and/or sensory attributes of the tobacco being assessed. The systems and method are also able to recognise and learn about new types of tobaccos.

In practical terms, the examples described herein can use HSI methods to assess the buying channels of tobacco samples to provide sensory information and links to other tobacco quality properties. Due to this, full quality information for each tobacco bale can be produced in real-time during the buying process. When the methods and systems are used in assessing packing channels, the real-time sensory information will provide a more effective quality control and, consequently, batch problems can be remedied in advance.

The systems and methods will now be described in greater detail with reference to the figures.

Prior to the analysis of a new, unknown tobacco sample, (i.e. a tobacco sample to be analysed) a prediction model, which is based on a first, reference spectral fingerprint dataset and a second, known property, dataset, is created. The first, reference spectral fingerprint dataset is created by scanning a plurality of known tobacco samples using HSI techniques. For example, in practice, reference datasets for the prediction model can be built individually for each of the buying channel and packing channel. The sample set could be composed of around 1500 samples, e.g. Virginia, Burley, Comum, Dark, Amarelinho from a particular crop year, e.g. from the 2019 or 2020 year crop. Other samples could of course alternatively be used to create the reference dataset. The required number of samples for modelling may be different depending on the property of interest. Thus it could be more or less than 1500 samples from one or more crops.

Images of these known samples are then acquired using a near infrared hyperspectral imaging (NIR HSI) system. External parameters such as luminosity, distance between camera and sample surface, and conveyor belt speed can be controlled to standardize image acquisition.

After acquiring the HSI data for the known, reference samples, the images may be corrected using a background and a reference spectrum for extraction of a near infrared fingerprint. Instrumental parameters and installation conditions of each SWIR camera are detailed in this Table 1 below,

| | | | |
|---|---|---|---|
| | Buying channel | Buying channel | Packing |
| Parameter | line 1 | line 2 | channel |

Instrumental conditions

| Frame rate (Hz) | 150 | | 150 | | 100 | |
|---|---|---|---|---|---|---|
| Exposure time (ms) | 4 | | 4 | | 7 | |
| Spectral binning | 1 | | 1 | | 1 | |
| Spatial binning | 1 | | 1 | | 1 | |
| Selected workflow | Image and dark | | Image and dark | | Image and dark | |
| | image | | image | | image | |
| Capture mode | Scripted | | Scripted | | Scripted | |
| Frame count | 250 | | 250 | | 250 | |
| Dark frame count | 10 | | 10 | | 10 | |
| Lens | 30 | mm | 30 | mm | 56 | mm |

| | | | |
|---|---|---|---|
| Calibration Set | OLE30 | OLE30 | OLE56 |

Installation conditions

| | | | | | | |
|---|---|---|---|---|---|---|
| Distance | 110 | cm | 110 | cm | 223 | cm |
| between camera | | | | | | |
| and conveyor | | | | | | |
| belt | | | | | | |
| Distance | 70 | cm | 70 | cm | 70 | cm |
| between camera | | | | | | |
| and sample | | | | | | |
| surface | | | | | | |
| Distance | 81 | cm | 79 | cm | 200 | cm |
| between lights | | | | | | |
| and sample | | | | | | |
| surface | | | | | | |
| Platform height | 40 | cm | 40 | cm | 40 | cm |
| Conveyor belt | 70 | cm | 70 | cm | 70 | cm |
| height | | | | | | |

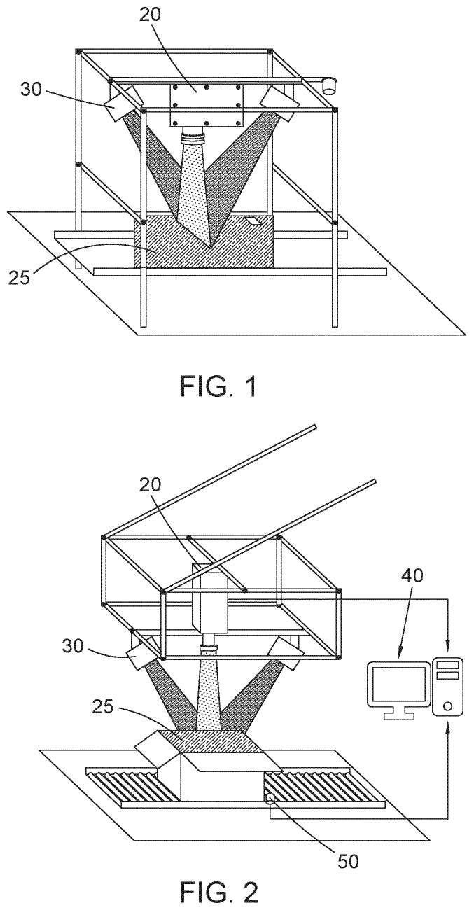

An illustrative scheme of the installation of the cameras in buying and packing channels is shown in FIGS. 1 and 2 respectively. As can be seen in those figures, the known sample 25 that is being analysed for the reference spectral fingerprint dataset is scanned via the HSI camera 20 under illumination 30. A detector 50 obtains information from the samples 25 and this information is input into the computer 40 which generates a reference spectral fingerprint of each of the known samples (and which eventually uses these to create a prediction model).

When generating these spectral fingerprints, particular components, properties and/or attributes may be analysed. For example, the total alkaloids, total sugar and nitrate of a plurality of samples from each of the samples may be determined by an automated flow injection system with UV/Vis detector. Properties may also include toxicants, or sensory attributes such as sweetness, irritation, among others.

For alkaloids, this may be based on the colouring reaction of the nicotine in the sample, sulphanilic acid and cyanogen chloride. For total sugars, the colouring reaction may result from the reduction of potassium iron cyanide by monosaccharides.

Each of the generated reference spectral fingerprints are then stored into the first reference spectral fingerprint dataset and this dataset is input into a machine learning-based prediction framework.

A second, known property, dataset is also created and input into the machine learning-based prediction framework. The second, known property, dataset may be related to known properties and/or composition(s) of said known tobacco samples. These known properties may be sensory attributes, taste profile, leaf position, leaf colour and/or leaf quality that may be determined by human assessment or by an indirect instrumental method obtained in a laboratory. In some embodiments the composition may be related to concentration levels of chemical compounds determined by analytical instrumental methods in a laboratory. The dataset is not, however, limited to only these properties but other properties may also be used.

The machine learning based prediction framework correlates the first, spectral fingerprint dataset with the known property or properties of the second, known property, dataset and generates a prediction model/models based on the first and said second datasets.

This step of correlating may comprise correlating the property of each of the reference spectral fingerprints with one or more of a chemical compound, a toxicant, determination of a leaf position, leaf colour, leaf quality, and/or a sensory attribute.

The prediction models are therefore generated based on the reference spectral fingerprint dataset and their respective reference properties (obtained from the correlation with the second, known property, dataset).

That is, the prediction models comprise the spectral fingerprint of the reference samples which are correlated to their specific features such as components and levels thereof, properties, sensory attributes etc.

Prediction models for specific chemical targets may be developed by the machine-learning based prediction framework by correlating and associating the near infrared spectral fingerprints of the known samples with chemical target reference values stored in the second, known property dataset. The prediction models may also be generated by correlating one or more properties of the spectral fingerprints of the known, reference samples with a sensory attribute or attributes of the tobacco sample as stored in the second, known property, dataset.

In some examples, the spectral fingerprints may be pre-processed using a standard normal variate (SNV) algorithm, Savitzy-Golay first derivative (windows with 15 points) and mean centering; and the extreme spectral variables may be excluded. The SNV algorithm removes multiplicative light effects that can occur because of light heterogeneity. The Savitzy-Golay first derivative removes baseline effects and shifts.

Then, the machine learning-based prediction framework generates and develops a partial least square (PLS) mathematical function (e.g. regression model) for each target (total alkaloids, total sugars, nitrate, matrix bound NNK, free NNK, free NNN, free NAT, and free NAB). Samples with high statistical residues (outliers) may be removed from the modelling as appropriate. The performance of the models may then be evaluated by cross-validation, external validation and permutation test. The models may be developed independently for each channel (e.g. buying and packing).

That is, the prediction model is based on the first, reference spectral fingerprint dataset of the known samples and the second, known property dataset, and is configured to determine one or more mathematical functions (e.g. regression coefficients) using these datasets (having known sensory values). As discussed further below, the prediction model is configured to apply these one or more mathematical functions (e.g. regression coefficients) to the spectral fingerprints of the unknown samples in order to quantify the sensory attributes, properties or features of the unknown samples.

A new, unknown tobacco sample may then be analysed based on its spectral fingerprint and prediction models described above. As in the case for the reference samples discussed above, a hyperspectral imaging camera such as those depicted in FIGS. 1 and 2 is also used to scan the unknown tobacco sample and hyperspectral images of the unknown tobacco sample are then generated.

The hyperspectral images of these new, unknown samples may be pre-processed to extract a near infrared spectral fingerprint of the samples. Variations relating to external conditions may be corrected by the background and blank spectrum, and all non-tobacco objects, such as conveyor belt, paper labels, rope, etc., may be removed by image processing. After extracting the near infrared spectral fingerprints from these new images, the spectra may be pre-processed for baseline and intensities correction, and the extreme variables may be excluded to remove noisier spectral regions.

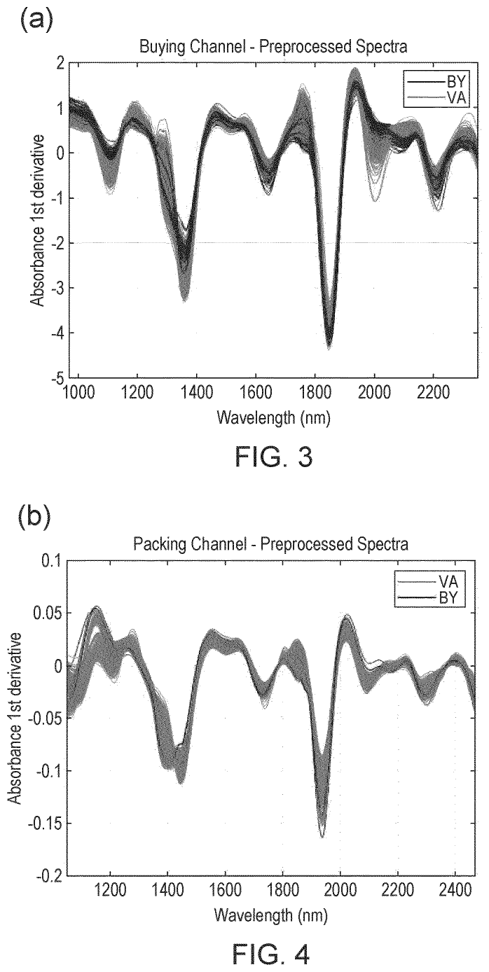

An example of spectral fingerprints of pre-processed spectra from buying and packing channels are shown in FIG. 3 and FIG. 4, respectively. The spectral profile difference between buying and packing channels is evident in these figures. Since the installation sets and physical characteristics of samples are quite different in the two channels, the analytical platform may use datasets dedicated for each channel, if desired.

The generated spectral fingerprint of the unknown tobacco sample is then input into the machine learning-based prediction framework.

The pre-processed spectra of the unknown sample that is being analysed is then associated with the respective reference values of the chemical constituent, via the machine based learning prediction framework, and using the prediction model. This is achieved by the prediction model being configured to apply the one or more mathematical functions (e.g. regression coefficients) to the new, unknown samples in order to quantify their sensory attributes. Due to this, a predicted sensory attribute of said the unknown tobacco sample may be outputted, based on said prediction model. The prediction models are applied to the pre-processed spectra of the unknown sample, via one or more mathematical functions (e.g. regression coefficients), to determine the unknown sample properties. Prediction models for chemical constituents output concentration of chemical constituents of the unknown sample; prediction models for sensory attributes output sensory attributes values of the unknown sample, etc.

In this way, the systems and methods described herein provide a computer implemented method of predicting a sensory attribute or attributes of an unknown tobacco sample using a prediction model that utilises one or more mathematical functions (e.g. regression coefficients).

In examples wherein the first, reference spectral fingerprint dataset comprises information relating to chemical components, or other properties of the tobacco sample, the systems and methods described herein may also provide a computer implemented method of determining such properties of an unknown tobacco sample, using the prediction model described herein. The properties may therefore comprise one or more of a chemical compound, a toxicant, determination of leaf position, leaf colour and/or leaf quality, taste profile and/or a sensory attribute of the unknown tobacco sample.

The systems and methods described herein also provide a computer implemented method of training a machine learning-based prediction framework for the determination of a property or properties of an unknown tobacco sample, the properties comprising one or more of a chemical compound, a toxicant, determination of leaf position, leaf colour and/or leaf quality, taste profile and/or a sensory attribute of said unknown tobacco sample.

The systems and methods described herein also provide a computer implemented method of training a machine learning-based prediction framework for the prediction of a sensory attribute or attributes of an unknown tobacco sample.

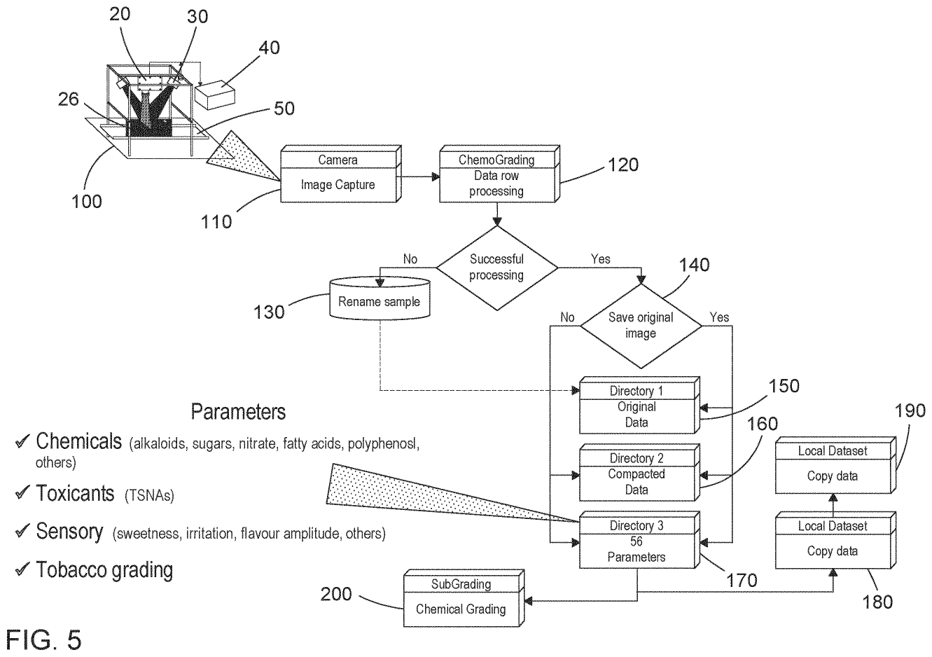

The possible flow of data in the tobacco assessment system and method described herein will now be described. FIG. 5 depicts the workflow of the application of prediction models in more detail. That is, FIG. 5 depicts a flow chart indicating the data flow that may be generated when the unknown tobacco sample 26 is being assessed by the prediction model.

In summary, the machine learning based prediction framework may be configured to train the prediction model based on the two datasets, i.e. the first, reference spectral fingerprint dataset (which comprises the spectral fingerprints of the known samples as discussed above) and the second, known property, dataset (i.e. reference values of the known samples).

As discussed in detail above, the first reference spectral fingerprint dataset may be created based on the images obtained by the HSI camera (i.e. instrumental responses) and their respective generated spectral fingerprints.

The second, known property, dataset may be related to known properties and/or composition(s) of the known tobacco samples (i.e. known reference values). These known properties may be sensory attributes and/or taste profiles that in some embodiments may be determined by human assessment or in other embodiments, by an indirect instrumental method obtained in a laboratory. In some embodiments, a mixture of the two may be used to generate this second, known property, dataset. The composition of the known tobacco samples may be related to concentration levels of chemical compounds determined by analytical instrumental methods in a laboratory.

As shown in FIG. 5, the tobacco sample that is to be assessed is inserted into a hyperspectral imaging machine 100. A hyperspectral imaging camera is used to scan the unknown tobacco sample 26 and generates hyperspectral images 110 of the unknown tobacco sample 26.

Based on the hyperspectral images that have been generated of the unknown tobacco sample, a spectral fingerprint of the unknown tobacco sample is then generated. This newly generated spectral fingerprint of the unknown tobaccos sample 26 is then input into the machine learning-based prediction framework, as discussed above.

The machine-based learning prediction framework is configured to utilise the prediction model for sensory attribute prediction by applying the one or more mathematical functions (e.g. regression coefficients) to the new unknown samples 26. The predicted results (chemical concentration, sensory attribute, or other property) are then generated.

In some examples, Chemograding software 120 may be used for image processing, application of models and export of predicted results.

The Chemograding is used for image processing, application of the models to new samples, and generation of results.

The generation of prediction models is performed using some IDE or software, such as Matlab. After developing or updating prediction models, they are included into Chemograding.

During the Chemograding step, the images collected by HSI of the known, reference samples may be pre-processed and the prediction model may be generated based on the reference datasets.

As discussed above, the prediction models may be trained using the first reference spectral fingerprint dataset which is built with raw or compacted images, processed or not processed images of the known tobacco samples 25, and the a second, known property, dataset which is built with references values (e.g., concentration of chemicals, sensory attributes) or known proprieties (e.g., tobacco grade) of the known samples.

As discussed above, the first reference spectral fingerprint dataset comprises the spectral fingerprints generated from the HSI images of the known samples. The modelling can be performed using the images in raw format (one spectrum per pixel) or in compact format (mean spectrum from all pixels). Moreover, in some examples, but not necessarily all, pre-processing algorithms may be applied to the images (in raw or compact format) to improve modelling performance.

The second, known property, dataset may comprise reference values of known samples. The reference values may be the concentration of chemical compounds, taste profile, sensory attributes, or tobacco grading etc.

The algorithms used to train the prediction models can be linear, non-linear, univariate or multivariate. The algorithm finds correlations between the first and second datasets, resulting in a function that correlates the instrumental response and sample components and/or properties. Finally, this function (trained model) can be applied to unknown samples to predict the components and/or properties.

In the embodiment shown in FIG. 5, the prediction models are built using the pre-processed compacted data and reference values (e.g., concentration of chemical compounds, sensory attributes, and grade) of known samples. One prediction model is built for each chemical compound and property. To apply the prediction models, the image of known or unknown samples must be compressed and pre-processed using the same steps used to build the models.

The prediction models are built using the above-discussed first reference spectral fingerprint dataset of HSI images and the second, known property, dataset of reference values of known samples. The HSI images can be used in modelling in raw or compact format, pre-processed or not pre-processed by algorithms.

In some examples, the prediction models may be built using the HSI images in the compact format (mean spectrum from all pixels) and pre-processed by algorithms (standard normal variate (SNV), Savitzy-Golay first derivative (windows with 15 points) and mean centering).

The software may be used in the embodiments described herein may be configured to segment the image, e.g. by separating tobacco for foreign materials, compile the pre-processed spectral fingerprint and perform the prediction through machine learning. In order to do this, the equipment acquires a raw image of the process. In this way, the software is therefore configured to transform the raw image into structured information, segment the tobacco material from the conveyor belt, build the spectral fingerprint with that and make the predictions.

During this step of Chemograding, the prediction model comprising one or more mathematical functions (e.g. regression coefficients) is applied to the unknown sample 26 and the machine learning software used in the systems and methods described herein may provide an output indicating the chemical grade of the unknown tobacco sample.

The machine learning approach may use univariate or multivariate linear algorithms, non-linear algorithms, or neural networks approaches. When performing the assessment of the chemical grade of the tobacco sample, the software may be configured to perform pre-processing of the image of the tobacco sample being assessed. It may also be used in Subgrading software, 200 to subgrade the sample and segregating the tobacco sample based on the chemical grade detected. This is discussed further below.

There is no need to save the processed data of unknown samples into a database for obtaining its grading. Processed data of unknown samples may be saved just for future proposes, but it is not a requirement for real-time grading analysis.

The processed data of the reference samples with known references values (e.g., chemicals, sensory, and grading results) are, however, saved to update the reference database. Then, machine learning can take place, since the prediction models can be trained using this saved processed data with their respective reference database. After training the models, they are applied to the processed data of further unknown samples through multiples coefficients, resulting in several outputs (chemicals, sensory, and grading predictions).

The chemical grading step 120 may determine the levels of components of certain chemicals in the sample, such as alkaloids, sugars, nitrates, fatty acids, polyphenol, as well as others. The sample can also or alternatively be analysed for toxicants or for determining its sensory attributes. Sensory attributes may be, for example, sweetness, irritation, amplitude, among others.

The final step of Chemograding 120 comprises performing chemical grading of that unknown sample, 26, using the spectral fingerprint, i.e. the processed image, for example. That is, in order to obtain the grading of an unknown sample, all the following steps may be performed: (1) Image processing; (2) transform the processed image to a compact data (fingerprint); (3) pre-process the fingerprint; and (4) apply the trained prediction models to generate the grading of the unknown sample 26.

Returning now to the discussion of the data flow shown in FIG. 5, if the processing is unsuccessful, for example, if the equipment captures an image that does not have any tobacco in it, or an image that contains an image with two tobacco samples in it, then the sample is renamed based on the output of that processing, 130 and saved in a database comprising the original data 150. If, the data processing is successful, 140, then the original image is saved in the database comprising also the original data without being renamed. This data may be saved as compacted data 160 and may also be saved for up to 56 properties 170. The processed data may be saved, for example, on a local server 180 in a local dataset 190 or by other means such as blog storage or cloud etc.

In some examples, Subgrading software 200 may also be used to optimize sourcing by creating new tobacco classes according to the user's criterion and assign the new classes to the samples according to the results generated by the Chemograding software. For example, if the user wants to select samples with low NNK content and a certain range of total alkaloids, a new digit will be created and added to the predicted internal class for samples that meet the criteria. The new classes created according to the new criterion can be used in GTL's internal system for selection of more specific classes.

Subgrading is therefore configured to segregate tobacco based on the Chemograding predictions. The software may be configured to read what was predicted by the previous model and compare that with the user rules input for grading and perform the reclassification. For example, the user may create rules based on chemicals such as the user may want tobacco with nicotine above 3%, with low amount of polyphenols. When the tobacco attends to these rules, the tobacco is segregated automatically (the grade is change for the new one, as well as their inputs in the system.)

The system and method described herein may therefore be described as being a chemical method that is coupled with machine learning algorithms that is configured to translate hyperspectral images of tobacco and chemical and sensory properties to grade or monitor it. The system uses machine learning to predict at least 56 tobacco properties based on the tobacco sample's chemical composition. 1 image (125 MB) can be processed in just 3 seconds. The size of the image file may be different, however, depending on the parameter settings of the camera. The processing may also demand more or less than 3 seconds depending on the settings of the camera parameters and the computer hardware. The prediction models that are used as reference databases are built using thousands of tobacco images. It may alternatively use more or less than this amount, depending on the property of interest. The tobacco samples used for this reference database can be taken from crops produced over a range of years. The models can also be built with just one crop as well, however, the use of more crops will result in models that are more robust and representative.

This system is therefore able to recognize new tobacco profiles. The properties of samples with new tobacco profiles (i.e., samples out of the models' scope) must be determined by reference methods (instrumental or human assessments). Then, these new samples can be added to the dataset, increasing it crop by crop.

The tobacco properties that are detected may comprise the following properties, amongst others: sucrose esters, diterpenes, fatty acids, polyphenols, carotenoids. Total alkaloids, total sugar, tobacco specific nitrosamines (TSNAs), taste profile, as well as other sensory attributes such as sweetness, bitterness, etc.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future.

1. A computer implemented method of determining a property or properties of an unknown tobacco sample, the method using hyperspectral imaging in combination with a machine-learning-based prediction framework,
the method comprising:
creating a first, reference spectral fingerprint dataset by scanning a plurality of known tobacco samples using hyperspectral imaging techniques, generating hyperspectral images of each of said known tobacco samples and forming a reference spectral fingerprint of each of said known samples, and storing each of said reference spectral fingerprints into said first, reference spectral fingerprint dataset,
providing a second, known property, dataset, comprising known properties and/or compositions of said known tobacco sample, and
inputting said first, reference spectral fingerprint dataset and said second, known property, dataset into said machine learning-based prediction framework
via said machine learning-based prediction framework, correlating said first, spectral fingerprint dataset with said known property or properties of said second, known property, dataset
said machine-learning based prediction framework generating a prediction model that is configured to determine one or more mathematical functions based on said first reference spectral fingerprint dataset, and said second, known property dataset,
using a hyperspectral Imaging camera to scan said unknown tobacco sample and generating hyperspectral images of said unknown tobacco sample, and based on said hyperspectral images of said unknown tobacco sample, generating a spectral fingerprint of said unknown tobacco sample,
inputting said spectral fingerprint of said unknown tobacco sample into said machine learning-based prediction framework, and
said machine-learning-based prediction framework applying said one or more mathematical functions of said prediction model to said spectral fingerprint of said unknown tobacco sample to obtain and output said determined property of said unknown tobacco sample based on said prediction model.

2. A computer implemented method of training a machine learning-based prediction framework for determining a property or properties of an unknown tobacco sample, the method comprising:
creating a first, reference spectral fingerprint dataset by scanning a plurality of said known tobacco samples using hyperspectral imaging techniques, generating hyperspectral images of each of said known tobacco samples and forming a reference spectral fingerprint of each of said known samples,
creating a second, known property, dataset, relating to a known property and/or composition of said known tobacco sample, and
inputting said first and second datasets into said machine learning-based prediction framework, and
via said machine learning based prediction network, correlating said first, spectral fingerprint dataset with said known property or properties of said second, known property, dataset and generating a prediction model based on said first and said second datasets, said machine-learning based prediction framework generating a prediction model based on said first and said second datasets, and
using a hyperspectral imaging camera to scan said unknown tobacco sample and generating hyperspectral images of said unknown tobacco sample, and based on said hyperspectral images of said unknown tobacco sample, generating a spectral fingerprint of said unknown tobacco sample,
inputting said spectral fingerprint of said unknown tobacco sample into said machine learning-based prediction framework, and
said machine-learning-based prediction framework applying said prediction model to said spectral fingerprint of said unknown tobacco sample to obtain and output said determined property of said unknown tobacco sample based on said prediction model.

3. The computer implemented method of claim 1 wherein said outputted property or properties comprises one or more of a chemical compound, a toxicant, determination of leaf position, leaf colour and/or leaf quality, taste profile, and/or a sensory attribute of said unknown tobacco sample.

4. The computer implemented method of claim 1, wherein said known properties of said second dataset comprise one or more of chemical compound, a toxicant, determination of leaf position, leaf colour and/or leaf quality, taste profile, and/or a sensory attribute of said unknown tobacco sample.

5. The computer implemented method of claim 1, further comprising said prediction model chemically grading said unknown tobacco sample based on said predicted property.

6. The computer implemented method of claim 5, further comprising said prediction model segregating said unknown tobacco sample based on said predicted property.

7. The computer implemented method of claim 1, further comprising said prediction model identifying, analysing, and/or quantifying chemicals contained within said unknown tobacco sample based on said predicted sensory attribute.

8. The computer implemented method of claim 7, further comprising said prediction model segregating said unknown tobacco sample based on said chemical quantification.

9. The computer implemented method of claim 1, wherein said machine-learning based prediction framework utilises linear algorithms, or wherein said machine learning based prediction framework utilises non-linear algorithms.

10. The computer implemented method of claim 1, wherein said machine-learning based prediction framework a neural network.

11. The computer implemented method of claim 1, wherein the method is implemented using a numerical computing environment and/or framework

12. The computer implemented method of claim 11, wherein said numerical computing environment and/or framework comprises any of MATLAB, GUI Octave, Pycharm, R Studio, and Anaconda.

13. The computer implemented method of claim 1, wherein said method is implemented using one or more programming languages.

14. The computer implemented method of claim 13, wherein said one more programming languages comprises any one or more of Python, C, C++, C#, R, and Java.

15. A machine learning-based prediction framework produced by the method of claim 2.

16. A computer system for producing a machine learning-based prediction framework, wherein the computer system is configured to perform the method of claim 2.

17. A computer system for determining a property or properties of an unknown tobacco sample, wherein the computer system is configured to perform the method of claim 1.
